(19) United States
(12) Patent Application Publication
(10) Pub. No.: US 2021/0261914 A1
(43) Pub. Date: Aug. 26, 2021

(54) ELECTROSPUN CELL SCAFFOLDS AND RELATED METHODS

(71) Applicant: Marshall University Research Corporation, Huntington, WV (US)

(72) Inventor: Nasim Nosoudi, Huntington, WV (US)

(21) Appl. No.: 17/180,331

(22) Filed: Feb. 19, 2021

Related U.S. Application Data
(60) Provisional application No. 62/979,027, filed on Feb. 20, 2020.

(51) Int. Cl. C12N 5/00; D01F 6/60; D01F 6/62; D01D 5/00; D01F 1/02; D01F 11/14; C12N 5/0775

(52) U.S. Cl. CPC C12N 5/0068; D01F 6/60; D01F 6/625; C12N 5/0663; D01F 1/02; D01F 11/14; C12N 5/0667; D01D 5/0007

(57) ABSTRACT

Cell scaffolds are provided comprising an electrospun fiber and one or more live cells that are incorporated directly into the electropsun fiber during an electrospinning process. The cell scaffold further include a protectant polymer that reduce damage to the cells during the electrospinning process and in which the live cells are embedded following electrospinning. Methods of making a cell scaffold including one or more live cells are further provided and comprise mixing one or more live cells with a protectant polymer and a biocompatible solvent to form a solution, and electrospinning the solution at a working voltage of about 8 kV to about 35 kV. Such methods can make use of a stem cell and a working voltage sufficient to differentiate the stem cell, including differentiation into a chondrocyte.

### RELATED APPLICATIONS

This application claims priority from U.S. Provisional Application Ser. No. 62/979,027, filed Feb. 20, 2020, the entire disclosure of which is incorporated herein by this reference.

### TECHNICAL FIELD

The presently-disclosed subject matter generally relates to electrospun cell scaffolds and related methods. In particular, certain embodiments of the presently-disclosed subject matter relate to electrospun cell scaffolds and methods for making the cell scaffolds by which live cells are electrospun and incorporated directly into the fibers of the scaffolds.

### BACKGROUND

Tissue engineering aims to produce synthetic tissues that can maintain, restore, or improve native tissue functions. Tissue engineering utilizes the formation of both acellular scaffolds as well as scaffolds that are seeded with cells. Acellular scaffolds are typically used to define an environment for new tissue to develop. These scaffolds will mimic the extracellular matrix, and promote cell adhesion and growth in vivo. Scaffolds with cells already seeded upon them are of increasing interest, as they are able to closely mimic human tissue. The production of these scaffolds greatly depends on the ability of the scaffold to allow for cell adhesion and migration. Scaffolds are usually porous and can be created by various means, such as electrospinning, phase-separation, freeze drying, and self-assembly. The ultimate goal of the creation of these scaffolds is to enhance the body's ability to heal itself, by providing a biodegradable matrix that can enable cells to grow.

In this regard, electrospinning is a quick and efficient way to produce scaffolds, and allows the control of many parameters of the scaffold, especially nanofiber and nanopore size. Other parameters can be determined as well, based on careful selection of a polymer and an appropriate solvent, as well as the electrospinning process itself. During electrospinning, the polymer is dissolved in an appropriate solvent and placed in a syringe. The syringe is then inserted into a syringe pump, which expels the polymer solution at a desired flow rate. In addition, a positive or negative lead is connected to the needle-tip of the syringe, while a ground lead is placed on a collector plate. The distance between the syringe-tip and the collector plate can be varied depending on the properties of the polymer solution and the applied voltage. When the electrostatic force on the polymer solution is enough to overcome the surface tension, a jet of polymer solution will form and eventually travel towards the collector plate. As the jet flows towards the collector plate, the liquid dissolves, leaving behind micro/nanofibers of the polymer, which strike the collector plate and produce the scaffold.

Utilizing current methods, cells are typically seeded onto such scaffolds after the scaffolds have been formed. Such cell seeding can be time consuming, however, as it requires three steps: creation of the scaffold, differentiation of the cells, and incorporation of the cells into the scaffold. Cell differentiation itself is time consuming and requires additional components, such as growth factors. Once these cells are differentiated and seeded another problem then arises, namely the limited ability of cell diffusion into the scaffold. Limited diffusion can produce a non-uniform distribution of cells that can cause varied properties and cell densities within different areas of the scaffold. That non-uniform distribution can then, in turn, be detrimental to the longevity of the scaffold both in vitro and in vivo. Accordingly, an improved method for incorporating cells into electrospun scaffolds would be both highly desirable and beneficial.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

In some embodiments of the presently-disclosed subject matter, a cell scaffold is provided that comprises an electrospun fiber and one or more live cells incorporated directly into the electropsun fiber. In some embodiments, the electrospun fiber is comprised of a protectant polymer. In some embodiments, the protectant polymer comprises a biocompatible water-soluble polymer, such as, in certain embodiments, a polysaccharide. In some embodiments, the protectant polymer is selected from the group consisting of poly(ethylene glycol) (PEG), polyvinyl pyrrolidine (PVP), polyvinyl alcohol (PVA), polyacrylic acid (PAA), polyethylene oxide (PEO), N-(2-hydroxypropyl methacrylamide (HPMA), polyoxazoline, dextran, xanthan gum, hyaluronic acid (HA), albumin, starch, pullulan, gelatin, and combinations thereof. In some embodiments, the polymer comprises gelatin, pullulan, or a combination thereof.

Turning now to the cells included in an exemplary cell scaffold of the presently-disclosed subject matter, in some embodiments, the one or more cells are selected from the group consisting of a stem cell or a macrophage. In some embodiments, the cells are stem cells such as, in some embodiments, an adipose-derived stem cell, a mesenchymal stem cell, or a bone marrow-derived stem cell.

Further provided, in some embodiments of the presently-disclosed subject matter are methods of making a cell scaffold that includes one more live cells incorporated directly into the cell scaffold. In some embodiments, a method of making a cell scaffold including one or more live cells comprises an initial step of mixing one or more live cells with a protectant polymer and a biocompatible solvent to form a solution. The solution is then electrospun at a working voltage of about 8 kV to about 35 kV such that an electropsun fiber is produced that incorporates the live cells directly into the electropsun fiber.

With further respect to the working voltages utilized in accordance with the presently-described methods, in some embodiments, the working voltage is about 8 kV to about 26 kV including, in some embodiments, a working voltage of about 8 kV or about 18 kV. In some embodiments, the one or more live cells incorporated into the scaffolds comprise a stem cell and the working voltage utilized is sufficient to differentiate the stem cell.

As indicated above, the protectant polymers utilized in accordance with the presently-disclosed subject matter can include a number of different biocompatible and/or water soluble polymers. In some implementations of the methods, such protectant polymers can further be combined with a biocompatible solvent, such as phosphate-buffered saline and/or cell culture media to further ensure and promote the viability of the live cells included in an exemplary scaffold. In some implementations of the methods, the protectant polymers and solvents included in the solution can further be mixed with one or more additional polymers. In some embodiments, the one or more additional polymers are selected from collagen, chitosan, poly(lactic-co-glycolic acid), and/or poly(ethylene oxide).

Still further provided, in some embodiments of the presently-disclosed subject matter, are methods of making a cell scaffold including one or more live chondrocytes. In some embodiments, a method of making a cell scaffold including one or more live chondrocytes is provided that comprises a step of mixing one or more stem cells with a protectant polymer and a biocompatible solvent to form a solution, and then electrospinning the solution at a working voltage of about 10 kV to about 20 kV.

Further features and advantages of the present invention will become evident to those of ordinary skill in the art after a study of the description, figures, and non-limiting examples in this document.

### BRIEF DESCRIPTION OF THE DRAWINGS

FIGS. 1A-1B include images showing exemplary electrospinning devices for spinning live cells in sterile conditions and made in accordance with the presently-disclosed subject matter;

FIGS. 2A-2B includes graphs showing (FIG. 2A) LDH release from attached cells in electrospun and control groups, and (FIG. 2B) LDH release from attached cells in electrospun (cell+media) compared to sprayed cell+media, where results are normalized to control;

FIG. 3 includes a graph showing gene expression in gelatin and gelatin/pullulan electrospun groups normalized to a control group;

FIGS. 4A-4B includes images showing actin staining of adipose derived stem cells in (FIG. 4A) control and in (FIG. 4B) pullulan/gelatin/cells at 10 kV, where FITC (green) labels actin and DAPI (blue) labels the nucleus, and showing (FIG. 4C) cells surrounded by FITC (green) conjugated gelatin; and

FIGS. 5A-5C includes images showing (FIG. 5A) cells stained with Cell Tracker green CMFDA (Invitrogen) at 2.5 μM for 1 hr prior to electrospinning and stained for DAPI after electrospinning, (FIG. 5B) a Cytoviva image of the cells and scaffold with no pre-staining, and (FIG. 5C) fourier-transform infrared spectroscopy (FTIR) of the pullulan, gelatin and gelatin/pullulan electrospun scaffolds.

FIG. 6 is a graph showing cell viability assessed by lactate dehydrogenase (LDH) measurement in cells 6 hours after electrospinning;

FIG. 7 is a graph showing a quantification of alcian blue staining in cells electropsun into a scaffold at 10 kV and 15 kV on day 7 (D7) and day 14 (D14) after electrospinning;

FIG. 8 includes images showing alcian blue staining in cells electropsun into a scaffold at 10 kV and 15 kV from day 2 (D2) to day 14 (D14) after electrospinning; and

FIG. 9 is a graph showing a volcano plot of RNA sequence analysis in cells electrospun into a scaffold 7 days after electrospinning and in control cells.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

The present application can “comprise” (open ended), “consist of” (closed ended), or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a,” “an,” and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently disclosed subject matter is based, at least in part, on the discovery that live cells can be incorporated directly into an electrospinning process, such that the cells are automatically spun into the scaffold without compromising cell viability and functionality of the cells. In some embodiments, a cell scaffold is provided that comprises an electrospun fiber and one or more live cells that are incorporated directly into the electropsun fiber.

The term “electrospun fiber,” and grammatical variations thereof, is used herein to refer to materials that are produced by an electrospinning process and are in the form of continuous filaments or discrete elongated pieces of material having diameters that are typically less than or equal to 1000 nanometers. Typically, such electrospinning techniques or processes, as indicated briefly above, make use of a high-voltage power supply, a spinneret (e.g., a hypodermic needle), and a collector (e.g., petri-dish). To perform the electrospinning process using these materials, an electrospinning liquid (i.e., a melt or solution of the desired materials that will be used to form the fibers) is generally first loaded into a syringe and is then fed at a specific rate set by a syringe pump. In some cases, a well-controlled environment (e.g., humidity, temperature, and atmosphere) can be used to achieve a smooth, reproducible operation of electrospinning.

As the liquid is fed by the syringe pump, at a desired voltage, the repulsion between the charges immobilized on the surface of the resulting liquid droplet overcomes the confinement of surface tension and then induces the ejection of a liquid jet from the orifice. The charged jet then goes through a whipping and stretching process, and subsequently results in the formation of uniform fibers. Further, as the jet is stretched and the solvent is evaporated, the diameters of the fibers can then be continuously reduced to a scale as small as tens of nanometers and, under the influence of electrical field, the fibers can subsequently be forced to travel towards the collector, onto which they are typically deposited. In this regard, by manipulating the electrical field or using mechanical force, different assemblies of fibers can be created. Moreover, in some embodiments, the fibers themselves can include various secondary structures, including, but not limited to, core-sheath structures, hollow structures, porous structures, and the like.

As noted, in some embodiments of the presently-disclosed subject matter, such electrospinning processes are utilized to incorporate live cells directly into the electrospinning solution, such that the resultant electropsun fibers include live cells that are incorporated directly into (e.g., embedded within) the electropsun fibers themselves. In particular, and without wishing to be bound by any particular theory or mechanism, it has been discovered that through the use of an electrospinning solution that includes a protectant polymer and a biocompatible solvent, electrospun fibers can be produced that incorporate live cells directly into the fibers without the concomitant loss of cell viability that typically occurs in electrospinning procedures that make use of harsh solvents or high voltage. In this regard, the term “protectant polymer” is used herein to refer to polymers that are capable of protecting and/or insulating cells from the voltage the cells may otherwise experience during an electrospinning process and that do not, by themselves, substantially affect the phenotype, viability, and/or differentiation of the cells. In some embodiments, such protectant polymers are comprised of a biocompatible, water-soluble polymer, such as a polysaccharide. In some embodiments, such protectant polymers include, but are not limited to, poly(ethylene glycol) (PEG), polyvinyl pyrrolidine (PVP), polyvinyl alcohol (PVA), polyacrylic acid (PAA), polyethylene oxide (PEO), N-(2-hydroxypropyl methacrylamide (HPMA), polyoxazoline, dextran, xanthan gum, hyaluronic acid (HA), albumin, starch, pullulan, gelatin, and combinations thereof. In some embodiments, the protectant polymer comprises gelatin, pullulan, or a combination thereof.

To produce an electrospinning solution in accordance with the presently-disclosed subject matter, which incorporates live cells, the protectant polymers are combined with a biocompatible solvent including the one or more cells to produce the electrospinning solution. As used herein, the term “biocompatible” thus refers to solvents not having toxic or injurious effects on the one or more cells to be included in the electrospinning solution. In some embodiments of the presently-disclosed subject matter, the solvent included in the electrospinning solution comprises water or a cell culture media (e.g., Dulbecco's Modified Eagle Medium (DMEM), Thermo Fisher Scientific, Waltham, Mass.).

Turning now to the cells included the cell scaffolds produced in accordance with the presently-disclosed subject matter, in some embodiments, the one or more cells that are included in an exemplary scaffold are cells that are appropriate for incorporation into a scaffold based on the intended use of that scaffold. For example, in some embodiments, cells that are appropriate for the repair, restructuring, or repopulation of a particular damaged tissue or organ will typically include cells that are commonly found in that tissue or organ or that can give rise to cells that are commonly found in that tissue or organ by differentiation or some other mechanism of action. In that regard, exemplary cells that can be incorporated into cell scaffolds of the presently-disclosed subject matter include stem cells, neurons, cardiomyocytes, myocytes, chondrocytes, pancreatic acinar cells, islets of Langerhans, osteocytes, hepatocytes, Kupffer cells, fibroblasts, myoblasts, satellite cells, endothelial cells, adipocytes, preadipocytes, biliary epithelial cells, and the like. These types of cells may be isolated and cultured by conventional techniques known in the art. Exemplary techniques can be found in, among other places; Freshney, Culture of Animal Cells, A Manual of Basic Techniques, 4th ed., Wiley Liss, John Wiley & Sons, 2000; Basic Cell Culture: A Practical Approach, Davis, ed., Oxford University Press, 2002; Animal Cell Culture: A Practical Approach, Masters, ed., 2000; and U.S. Pat. Nos. 5,516,681 and 5,559,022.

In some embodiments of the presently-disclosed subject matter, the cells included in an exemplary scaffold are stem cells. As used herein, the term “stem cells” refers broadly to traditional stem cells, progenitor cells, preprogenitor cells, precursor cells, reserve cells, and the like. Exemplary stem cells include, but are not limited to, embryonic stem cells, adult stem cells, pluripotent stem cells, induced pluripotent stem cells, neural stem cells, liver stem cells, muscle stem cells, muscle precursor stem cells, endothelial progenitor cells, bone marrow stem cells, chondrogenic stem cells, lymphoid stem cells, cardiac stem cells, mesenchymal stem cells, hematopoietic stem cells, central nervous system stem cells, peripheral nervous system stem cells, and the like. Descriptions of stem cells, including methods for isolating and culturing them, may be found in, among other places, Embryonic Stem Cells, Methods and Protocols, Turksen, ed., Humana Press, 2002; Weisman et al., Annu. Rev. Cell. Dev. Biol. 17:387-403; Pittinger et al., Science, 284:143-47, 1999; Animal Cell Culture, Masters, ed., Oxford University Press, 2000; Jackson et al., PNAS 96(25):14482-86, 1999; Zuk et al., Tissue Engineering, 7:211-228, 2001; and U.S. Pat. Nos. 5,559,022, 5,672,346 and 5,827,735. One of ordinary skill in the art will understand that the stem cells that are selected for inclusion in a scaffold are typically selected when such cells are appropriate for the intended use of a particular construct.

In some embodiments of the cell scaffolds described herein, the cells that are incorporated into the electrospinning solution and, in turn, the cell scaffolds are selected from a stem cell, a lymphocyte (e.g., a cancerous B lymphocyte), cancer cells, or a macrophage. In some embodiments, the stem cells are adult stem cells, such as, in some embodiments, adipose-derived stem cells or bone marrow-derived stem cells. In some particular embodiments, the adult stem cells are adipose-derived stem cells, as such adipose derived stem cells have been surprisingly found to be particularly useful in the cell scaffolds of the presently-disclosed subject matter.

In some embodiments, in addition to incorporating one or more cells within the scaffolds, various additional materials and/or biological molecules can also be attached to or used to coat the nanofiber scaffolds, either by direct encapsulation of the materials inside of the nanofibers during the electrospinning process or by post-modification procedures such as surface physical adsorption, surface chemical conjugation, and surface deposition. For example, in some embodiments, to improve the adherence and incorporation of a cell scaffold to a damaged tissue, an extracellular matrix protein, such as, in some embodiments, fibronectin, laminin, and/or collagen, is further attached to the nanofiber scaffold. As another example, in some embodiments where the nanofiber scaffold is to be used to replace or repair damaged heart or nerve tissue or other electrically-conductive tissue, the cell scaffold is coated or mixed with an electrically-conductive material, such as electrically-conductive polymer, a metal nanoparticle, or both.

As another example of materials that can be attached to or used to coat the cell scaffolds, in some embodiments, a growth factor is further attached to the nanofiber scaffold or one or more cells incorporated into the scaffold are transformed and made to express a growth factor to facilitate the repair and regeneration of the damaged tissue. In some embodiments, the growth factor is selected from the group consisting of vascular endothelial growth factor (VEGF), basic fibroblast growth factor (bFGF), insulin-like growth factor (IGF), placental growth factor (PIGF), Angl, platelet derived growth factor-BB (PDGF-BB), and transforming growth factor β (TGF-β). In some embodiments, the growth factor is VEGF.

In some embodiments of the presently-disclosed subject matter, a therapeutic agent (i.e., an agent capable of treating damaged tissue) is further attached to an exemplary cell scaffold. In some embodiments, the therapeutic agent is an anti-inflammatory agent or an antibiotic. Examples of anti-inflammatory agents that can be incorporated into the scaffolds include, but are not limited to, steroidal anti-inflammatory agents such as betamethasone, triamcinolone dexamethasone, prednisone, mometasone, fluticasone, beclomethasone, flunisolide, and budesonide; and non-steroidal anti-inflammatory agents, such as fenoprofen, flurbiprofen, ibuprofen, ketoprofen, naproxen, oxaprozin, diclofenac, etodolac, indomethacin, ketorolac, nabumetone, sulindac tolmetin meclofenamate, mefenamic acid, piroxicam, and suprofen.

Various antibiotics can also be employed in connection with a cell scaffold made in accordance with the presently-disclosed subject matter including, but are not limited to: aminoglycosides, such as amikacin, gentamycin, kanamycin, neomycin, netilmicin, paromomycin, streptomycin, or tobramycin; carbapenems, such as ertapenem, imipenem, meropenem; chloramphenicol; fluoroquinolones, such as ciprofloxacin, gatifloxacin, gemifloxacin, grepafloxacin, levofloxacin, lomefloxacin, moxifloxacin, norfloxacin, ofloxacin, sparfloxacin, or trovafloxacin; glycopeptides, such as vancomycin; lincosamides, such as clindamycin; macrolides/ketolides, such as azithromycin, clarithromycin, dirithromycin, erythromycin, or telithromycin; cephalosporins, such as cefadroxil, cefazolin, cephalexin, cephalothin, cephapirin, cephradine, cefaclor, cefamandole, cefonicid, cefotetan, cefoxitin, cefprozil, cefuroxime, loracarbef, cefdinir, cefditoren, cefixime, cefoperazone, cefotaxime, cefpodoxime, ceftazidime, ceftibuten, ceftizoxime, ceftriaxone, or cefepime; monobactams, such as aztreonam; nitroimidazoles, such as metronidazole; oxazolidinones, such as linezolid; penicillins, such as amoxicillin, amoxicillin/clavulanate, ampicillin, ampicillin/sulbactam, bacampicillin, carbenicillin, cloxacillin, dicloxacillin, methicillin, mezlocillin, nafcillin, oxacillin, penicillin G, penicillin V, piperacillin, piperacillin/tazobactam, ticarcillin, or ticarcillin/clavulanate; streptogramins, such as quinupristin/dalfopristin; sulfonamide/folate antagonists, such as sulfamethoxazole/trimethoprim; tetracyclines, such as demeclocycline, doxycycline, minocycline, or tetracycline; azole antifungals, such as clotrimazole, fluconazole, itraconazole, ketoconazole, miconazole, or voriconazole; polyene antifungals, such as amphotericin B or nystatin; echinocandin antifungals, such as caspofungin or micafungin, or other antifungals, such as ciclopirox, flucytosine, griseofulvin, or terbinafine.

With further respect to the electrospinning of the scaffold in accordance with the presently-disclosed subject matter, to produce a cell scaffold, the fibers incorporating the one or more cells are typically electropsun at a working voltage of about 8 kV to about 35 kV such that the electrospinning process does not have a significant effect on the viability of the cells that are incorporated into the fibers. For instance, in some embodiments, the working voltage is about 10 kV, about 15 kV, about 20 kV, about 25 kV, about 30 kV, or about 35 kV. In some embodiments, the working voltage is about 8 kV, while, in other embodiments, the working voltage is about 18 kV. In yet further, embodiments, however, where the one or more cells being incorporated into the electropsun scaffold are stem cells, electrospinning the solution comprises electrospinning the solution at a working voltage sufficient to differentiate the stem cell as it has been surprisingly discovered that electrospinning at certain voltages (e.g., higher voltages such as 18 kV) induces the differentiation of stem cells, including the expression of cellular markers (e.g., neural markers such as Tuj-1 or chondrocyte marker such as markers in the TGF-β pathway) without the use of growth factors.

With that in mind, further provided by the presently-disclosed subject matter, in some embodiments, are methods of making a cell scaffold including one or more live chondrocytes. In some embodiments, a method of making a cell scaffold including one or more live chondrocytes is provided that comprises a step of mixing one or more stem cells with a protectant polymer and a biocompatible solvent to form a solution, and then electrospinning the solution at a working voltage of about 10 kV to about 20 kV.

In some embodiments, and in addition to electrospinning at various voltages to provide different types of scaffolds including live cells, different cell scaffolds including one or more live cells can also be produced by varying the type of electrospinning process utilized. For example, in some embodiments, uniaxial electrospinning processes may be utilized in which an electrospinning solution is utilized that includes a protectant polymer that is directly mixed with the one or more cells and biocompatible solvent (e.g., cell media). In other embodiments, and as another example, a coaxial electrospinning process can be utilized in which the electrospinning solution including the protectant polymer, the one or more cells, and the biocompatible polymer is further combined with an additional polymer solution before electrospinning. In some embodiments, such additional polymers can be selected from polymers such as, but not limited to, collagen, chitosan, poly(lactic-co-glycolic acid), and/or poly(ethylene oxide).

The presently-disclosed subject matter is further illustrated by the following specific but non-limiting examples. The following examples may include compilations of data that are representative of data gathered at various times during the course of development and experimentation related to the present invention.

### EXAMPLES

### Example 1—Incorporation of Viable Cells into Electrospun Scaffolds

Externally applied magnetic fields can affect cell differentiation, and it is likely that the generated electric field effects the cell membrane. In particular, when the cell membrane is forced to change shape, it is believed to, in turn, distort the cytoskeleton of the cell, which attaches the cell membrane to the nucleus. That change in the cytoskeleton then affects the expressed genes, brings about the creation of different cell signals, which could then induce differentiation. In that regard, and without wishing to be bound by any particular theory or mechanism, it was believed that incorporation of stem cells into an electrospinning process would expose the stem cells to an electric field, likely inducing unique behaviors of cells.

One concern of such a process was that the cells themselves would be unable survive the voltage used in electrospinning. While an electric field could cause unique behaviors, too large of an electric field could be detrimental for the cells. By acting on the cell membrane as previously described, specific protein channels in the membrane could be denatured, causing irreparable cell damage. To prevent this from occurring, it was believed that voltages should be kept as low as possible as typical electrospinning voltages range from 1 kV to 30 kV and as the required applied voltage to create a scaffold varies depending on the polymer used.

With that in mind, and as described below, experiments were initially undertaken to test different polymer combinations: collagen and poly(ethylene oxide) (PEO), gelatin and pullulan. These polymer combinations would be electrospun at around 8 kV. One other restraint for these experiments was the solvent used for dissolving the polymer. In current electrospinning methods, common solvents include acetic acid, dichloromethane (DCM), acetone, water, chloroform, and ethanol. These solvents could be toxic given direct incorporation of cells into the polymer-solvent solution. To overcome this restriction, cell media was used as the solvent.

Collagen was chosen as the initial polymer, as it is the primary constituent of the body's natural extracellular matrix. However, collagen is typically electrospun with acetic acid as the solvent, which would likely cause cell death. No studies have been done, however, to show the success of electrospinning collagen with cell media as the solvent, and therefore other polymers would also be utilized. Gelatin is denatured collagen; therefore, it had the possibility to create scaffolds with the same success as collagen. As previous studies had determined, PEO increases the yield of uniform fibers when electrospun with other polymers, so it was decided to use PEO as well. Pullulan and gelatin are commonly used together in hydrogels, and pullulan has been shown to have antioxidant potential. Based on this, a combination of pullulan and gelatin or pullulan or gelatin alone was also used for electrospinning. Adipose-derived stem cells (ADSCs) were selected for initial experiments, as those cells were easily obtained and have the potential to give rise to various terminally differentiated cells, such as osteoblasts, chondrocytes, adipocytes, and neurons. The cells were directly incorporated into five polymer solutions prior to electrospinning.

Materials and Methods.

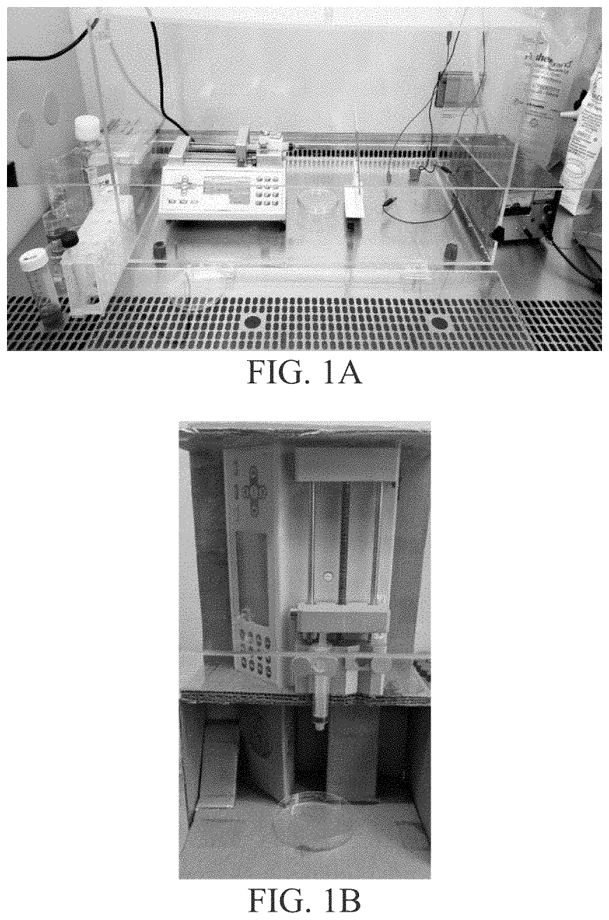

Electrospinning device. Due to the nature of working with living stem cells, it was imperative to maintain sterile conditions throughout the entire spinning process. In order to maintain a sterile environment, it was determined that spinning should take place under a sterile biological safety cabinet. The electrospinning device needed to withstand exposure to UV light, so that it could be sterilized for at least 24 hours. Acrylic, which can handle UV exposure, was determined to be the material with which the electrospinning device would be constructed. Acrylic sheets and cement were used to construct the framework of the electrospinning device, along with the necessary spinning supplies such as a plate, voltage supply, electrical leads, and syringe pump. The electrospinning device (FIG. 1A or 1B) was then placed under UV light within the safety cabinet for proper sterilization.

Cell Culture. P2-P4 of human adipose tissue-derived stem cells (hASCs) from Lonza (Walkersville, Md., USA) were used for cell culture. Cells were plated in T75 culture-treated flasks at about 1 million cells per flask. Culture media was changed every 3-4 days for the duration of the culture.

Electrospinning. As shown in Table 1 below, using 3 different protectants, 7 different solvents (including no-solvent), and changing voltage with increments of 2 gave 210 independent experiments to run at one time point. Looking at 4 different time points of D1, D7, D14 and D21 gave 840 experiments to run. The 8-26 kV range of voltages was chosen because in preliminary experiments 8 kV was the minimum voltage that could be used for electrospinning and 26 kV was the voltage where the viability of cells dropped to 50% even with a protectant.

TABLE 1
Cell Electrospinning Trials.

| | | | | Working |
|---|---|---|---|---|
| | | | | voltage |
| Protectant | Solvent | Cells | Electrospinning | (kV) |
| Pullulan | None | hASCs | Uniaxial | 8-26 |
| Gelatin | None | hASCs | Uniaxial | 8-26 |
| Poly(2-oxazoline)s | None | hASCs | Uniaxial | 8-26 |
| Pullulan | Collagen, chitosan, PLGA, PEO | hASCs | Coaxial | 8-26 |
| Gelatin | Collagen, chitosan, PLGA, PEO | hASCs | Coaxial | 8-26 |
| Poly(2-oxazoline)s | Collagen, chitosan, PLGA, PEO | hASCs | Coaxial | 8-26 |

As noted, P2-P4 of adipose tissue-derived stem cells (hASCs) from Lonza (Walkersville, Md., USA) were used for cell culture. Cells were plated in T75 culture-treated flasks at about 1 million cells per flask and culture media was changed every 3-4 days for the duration of the culture. Three components made up the cell electrospinning solution: protectant, solvent, and cell pellet. Collagen, poly(ethylene oxide), pullulan, and gelatin powders were used as the protectants. Poly(ethylene oxide) (Sigma), pullulan (Hayashibara Laboratories, Okayama, Japan), type A gelatin from porcine skin (Electron Microscopy Sciences, Hatfield, Pa.), and extracted collagen from rat tail were dissolved in solvent at concentrations of 2.5 mg/ml, 5 mg/ml, 10 mg/ml, 20 mg/ml, and 30 mg/ml. The protectants and mesenchymal stem cell medium (solvent) were mixed at the ratio of 1:1 (w/w) and placed on a stirring hot plate for 20-30 min to warm and mix. Solution was warmed to 40° C. in the case of gelatin. Tube temperature reduced to 37° C. Cell pellet was then added to the protectant solution. Cell electrospinning content was aseptically transferred to a sterile 10 ml syringe and a sterile 18-gauge syringe needle tip was secured. The collector plate, which was a petri dish, was positioned 3 inches from the end of the needle tip. The syringe pump settings were adjusted to produce readings for a plastic 10 ml syringe pump. The pump rate was set to 30 μl/min and reduced at increments of 5 μl/min to determine the optimized pump rate for each cell electrospinning solution. Control cells were sprayed at the same rate on the petri dish without any voltage application.

Viability test. The viability was investigated by live/dead assay kit and fluorescence microscopy. 6 hours after electrospinning, the culture media was aspirated from each well. After incubation with calcein and ethidium (2 μM calcein and 4 μM ethidium in PBS) for 10 minutes at 37° C., samples were washed with PBS and cells were imaged.

Cytotoxicity Test (lactate dehydrogenase (LDH) activity). Two days after spinning, media was aspirated, and cells were washed with PBS. Lactate dehydrogenase or LDH (Cytotox96 kit, Promega, Madison) was performed according to the manufacture's protocol to look at the cell viability using cell lysate.

$$Viability \% \frac{\_Average OD of sample * 100}{Average OD of control}$$

Gene expression by reverse transcription polymerase chain reaction (RT-PCR). 7 days after spinning, RNA was isolated according to the manufacturer's instructions for the RNeasy plus mini kit (Qiagen, USA), and RT-PCR was performed according to the instruction manual of the One-Step RT-PCR kit (Qiagen, USA). The selected pluripotential genes in the initial analysis were SOX2 and OCT4.

Immunocytochemistry. Cellular morphology was visualized at Day 2 using fluorescence microscopy. Briefly, cells were fixed with 4% paraformaldehyde (PFA) in PBS (pH 7.4) for 15 min at room temperature (RT). After rinsing with PBS for three times, the samples were placed in a permeabilization solution with 0.1% (v/v) Triton X-100 for 10 min and rinsed again with fresh PBS for three times. The cells were incubated with Phalloidin 488 and DAPI (Life technologies, Carlsbad, Calif.) to visualize the f-actin and nuclei, respectively.

Microscopy. To confirm the presences and distribution of cells within the protectant, FITC-conjugated gelatin along with DAPI stained cells were used. Electrospun cells/scaffold deposited on microscope glass slides were imaged using an Olympus BX51 microscope equipped with an Olympus DP73 camera and CellSens software.

To confirm that the cells were embedded within the scaffold, the cells were pre-labeled using a green CMFDA cell tracker dye (Invitrogen, Oreg., USA) prior to electrospinning, and then labeled with DAPI afterward. Samples were imaged using CytoViva's patented enhanced darkfield transmitted light condenser (NA 1.2-1.4) coupled with CytoViva's proprietary Dual Mode Fluorescence (DMF) module. These components were configured on an Olympus BX51 upright microscope using an Olympus100× oil UPL Fluorite objective (NA 0.60-1.30) with adjustable iris objective optimized for darkfield imaging. Light source used was Prior Lumen 200 with metal halide lamp and variable light attenuation. Optical images were captured using a DAGE-MTI XLMCT cooled CCD camera with 7.4 μm pixel size.

Fourier-Transform Infrared Spectroscopy (FTIR). Scaffold compositions were determined by loading onto an attenuated total reflectance (ATR) attachment and using a Thermo Scientific Nicolet iS 50 FTIR (Thermo Fisher, Waltham, Mass., USA). Data was plotted in MS Excel (Microsoft, Redmond, Wash., USA).

Results

Electrospinning was observed at a concentration of 5 mg/ml at 8 kV. Cells were detected 6 hours after electrospinning to observe attachment as a sign of viability. Most cells in collagen scaffold were dead (stained red). PEO scaffold had a lot of red cells floating in the petri dish. Gelatin, pullulan and pullulan/gelatin had good cell viability (stained fluorescent green), while the number of dead cells (stained red) was minor.

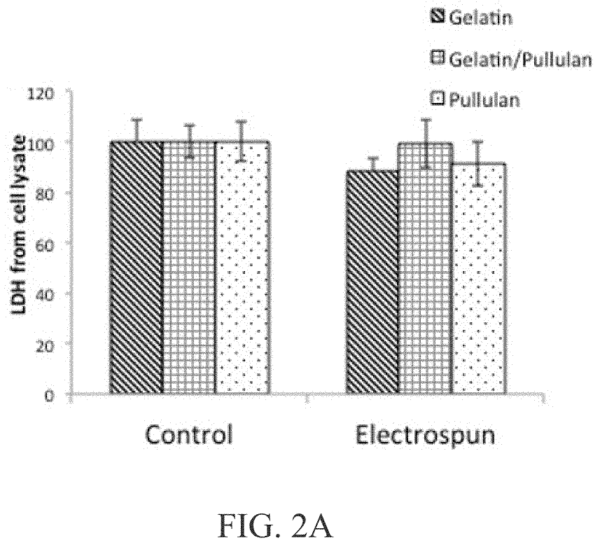

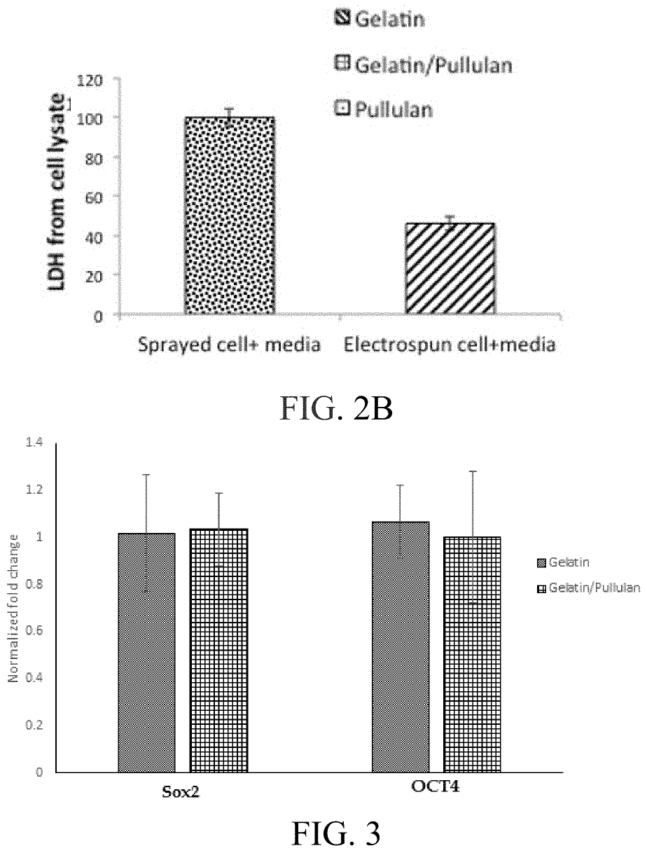

Viability of cells in collagen was very low in both control and electrospun groups. Control cells were just sprayed with the same rate on the petri dish without any voltage application. 0.01% acetic acid that was used to dissolve collagen was most probably the reason for low cell viability. PEO was dissolved in cell media and was biocompatible. However, very low cell attachment was observed in both the control and electrospun group. In the electrospinning process, the polymer solution is exposed to shear stress and cell death in the PEO group could be the result of non-Newtonian fluid behavior and shear stress. When PEO was removed from the formulation, gelatin/cell viability and attachment was improved and an analysis of LDH in cell lysate showed 88% cell viability of electrospun compared to the control (FIGS. 2A-2B). However, when switched to a combination of pullulan/gelatin/cells, 99% viability was achieved as compared to the control, although pullulan/cell scaffold itself had 91% viability. To prove the role of the protectants (gelatin and pullulan), cells have been electrospun with just culture media and cell viability reduced to 40% compared to cells and media that were just sprayed with the same rate on the petri dish.

Seven (7) days after electrospinning oil red O, toluidine blue, and alizarin red S staining was used to study adipogenic, chondrogenic, and osteogenic differentiations. All cells were negative for oil red O, toluidine blue, and alizarin Red S. Moreover, PCR data showed no significant change in SOX2 and OCT4 after electrospinning (FIG. 3) confirming stemness before and after electrospinning.

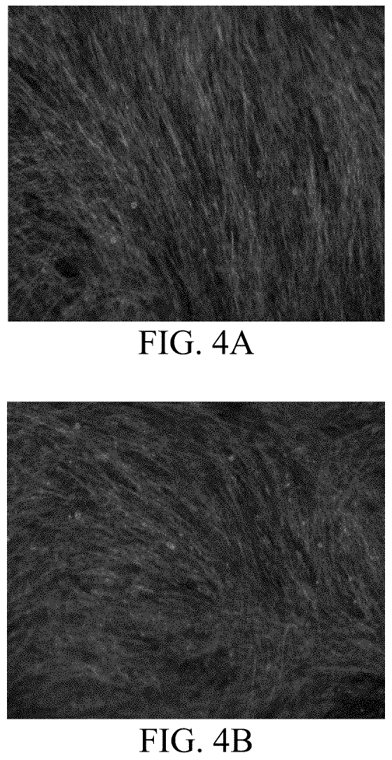

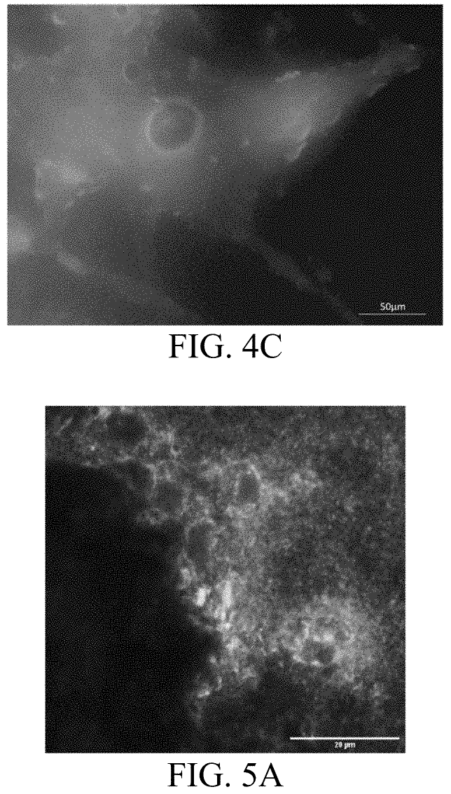

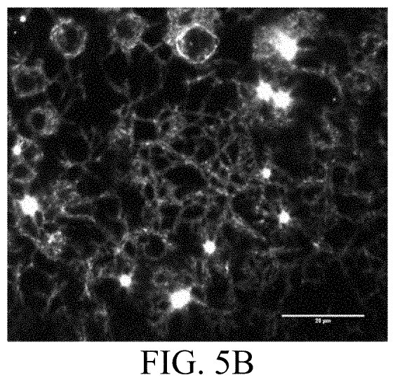

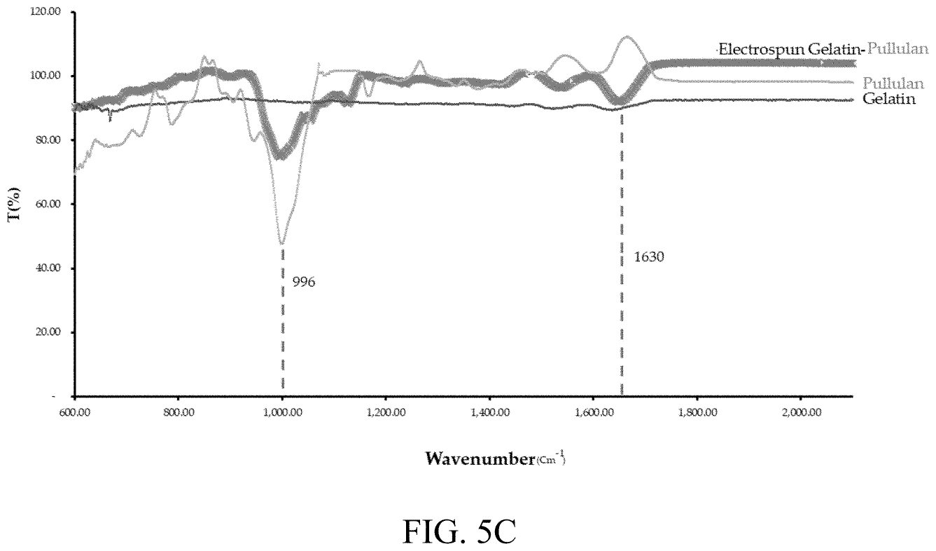

To look at the cell alignment, actin staining was used 2 days after electrospinning. Cell alignment was random as expected (FIGS. 4A-4B). Images of the scaffold with FITC gelatin and DAPI stained cells showed that cells were surrounded by green gelatin (FIG. 4C). A highly porous structure was observed after Cytoviva imaging. It appeared that the cells were embedded in these pores as confirmed by another Cytoviva imaging where cells were pre-stained with Cell tracker and DAPI. These pores that houses the cells appeared to have dimension of about 10 μm. (FIG. 5A-5B).

In the FTIR studies, the band at 996 cm⁻¹ was observed in Pullulan and electrospun samples, which was associated with C—OH bending vibrations at the C-6-position in the case of polysaccharide and indicates the strength of the interchain interactions via hydrogen bonding.

The primary hydroxyl groups at the C-6-position were available in the pullulan macromolecule (FIG. 5C). However, there were no hydroxyl groups at the C-6-position in gelatin. This band can show the glycosylation between the gelatin and pullulan molecules or formation of the interchain hydrogen bond in the composite fiber. The amide I (AmI) band at 1630 cm⁻¹ in pullulan/gelatin was strongest among the three and slightly shifted to a higher wavelength, which can be associated with AmI sensitivity to hydrogen bonding at the C═O group formation of triple helix state. Hydrogen bonding plays a significant role in stabilization of protein secondary structure which can be because of pullulan presence here.

Moreover, RNA was isolated according to the manufacturer's instructions for the RNeasy plus mini kit (Qiagen, USA), and RT-PCR was performed according to the instruction manual of the One-Step RT-PCR kit (Qiagen, USA) to determine the ability of the electrospinning process to differentiate the stem cells. The initial selected pluripotential genes were: SOX2 and OCT4. PCR data showed no significant change in SOX2 and OCT4 after electrospinning and confirmed stemness before and after electrospinning at 8 KV. To look at the cell alignment, actin staining was used 2 days after electrospinning. Cell alignment was random as expected. Interestingly, however, at higher voltages (18 kV), hASCs were observed to express Tuj-1, a neural marker. Although the viability at that this voltage was 70%, it appeared that the cells were differentiating without any growth factors.

Discussion

Uniaxial electrospinning using a single needle is a technology for the fabrication of scaffolds that can provide the initial scaffold for tissue engineering applications. Coaxial electrospinning on the other hand facilitates the incorporation and preservation of bioactive substances, whereas the shell was often used to protect sensitive substances encapsulated in the core. In the above-described method, uniaxial electrospinning was used while incorporating live cells. Polymers were used to protect the cells and the cells were encapsulated in the polymers. In this study, pullulan, gelatin, collagen, and PEO were used. Use of collagen and PEO was not overly successful and cell viability was not generally acceptable. Highly hydrated polymers like PEO suppresses cellular and molecular adhesions by providing a physical steric barrier.

In the study, it was also shown pullulan and gelatin could protect the cells from high voltage damages. Pullulan and gelatin were biocompatible water-soluble polymers that have been shown to be ineffective in changing phenotype, viability, and differentiation cells. Pullulan had the ability to quench reactive oxygen species and can be a great scaffold in combination with gelatin. Moreover, pullulan can increase the tensile strength of gelatin and that can be very important in tissue engineering. Its structural features, like the presence of large amounts of hydroxyl groups in the main chain, makes it a preferred polymer for making scaffolds. Studies show with the increase in pullulan content for scaffold leads to increase in viscosity and eventually leads to decrease in the electrical conductivity. Without wishing to be bound by any theory, however, it is believed that the composition that was being used for making scaffolds acted as a shield for live cells against electrical conductivity that is shown by viability studies.

SEM images showed fibers and cells (beads). Bead formation is often undesirable in electrospinning but in this case can accommodate the cells. Moreover, single cells covered by pullulan and gelatin were observed, which was confirmed by fluorescence microscopy. It has been shown that integrin is an electric field sensing protein on cell surface. On the other hand, gelatin attaches to cells via integrin. Blocking the sensing proteins may be the reason for protecting the cells from high voltage damages. In general, gelatin can protect the cells by covering the essential motifs required for cell function and viability.

In parts of the study, no significant difference was found in gene expression before and after electrospinning, but increasing the voltage and polymers appeared to change those results which is consistent with low voltage electrical stimulation affecting gene expression of transforming growth factor-β (TGF-β), collagen type-I, alkaline phosphatase (ALP), bone morphogenetic proteins (BMPs), and chondrocyte matrix.

In short, the above-described studies opened a new field within tissue engineering. The discovery that cells can be directly incorporated into the electrospinning process has many potential benefits within the tissue engineering realm.

### Example 2—Differentiation of Electrospun Cells

To further assess the ability of electrospinning conditions to induce cell differentiation, additional experiments were undertaken as described below.

Materials and Methods

Cell Culturing and Electrospinning. P2-P4 of adipose tissue-derived stem cells (hASCs) from Lonza (Walkersville, Md., USA) were used for cell cultures. Cells were plated in T75 culture-treated flasks with approximately 1 million cells per flask, and culture media was changed every 3-4 days for the duration of the culture. The gelatin/pullulan solution with the final concentration of 1.25 mg/ml was used for electrospinning. Cell electrospinning content was aseptically transferred to a sterile 10 ml syringe, and a sterile 18-gauge syringe needle tip was secured. The collector plate, which is a petri dish (Fisher brand, polystyrene), was positioned 7.5 cm from the end of the needle tip. The syringe pump settings were adjusted to produce readings for a plastic 10 ml syringe pump. The pump rate was set to 200 μL/min. Electrospraying was performed at 10 and 15 kV. Control experiment was performed without applying any voltages.

Viability Test. The viability was investigated by a live/dead assay kit and fluorescence microscopy. Approximately 6 hours after electrospinning, the culture media was aspirated from each well. After incubation with calcein and ethidium (2 μM calcein and 4 μLM ethidium in PBS) for 10 minutes at 37° C., samples were washed with PBS and cells were imaged.

Cytotoxicity Test (Lactate Dehydrogenase (LDH) Activity). The media was aspirated two days after spinning, and cells were washed with PBS. Lactate dehydrogenase or LDH (Cytotox96 kit, Promega, Madison) was performed on the attached cells according to the manufacturer's protocol to look at the cell viability using cell lysate. Again, Viability Percentage was calculated as the % Average OD of sample/Average OD of control.

Immunocytochemistry and histology. For the chondrocyte proteoglycan examination, cells in the petri dishes were fixed in 4% formaldehyde followed by staining with One percent Alcian Blue in 3% acetic acid.

Analysis of Glycosaminoglycan (GAG) content. On days 14 and 21, the cell culture was washed in PBS before being fixed using an acetone:methanol (1:1) solution at 4° C. for 3 min. One percent Alcian Blue in 3% acetic acid was added into the cell culture. The cells were incubated for 30 min and the overstaining dye was washed in 3% acetic acid and deionized water. One percent of sodium dodecyl sulfate (SDS) was added to the cell culture and homogenized using a shaker at 200 rpm for 30 min. The absorbance was read using a microplate reader at 605 nm wavelength. The observation was repeated three times.

Results

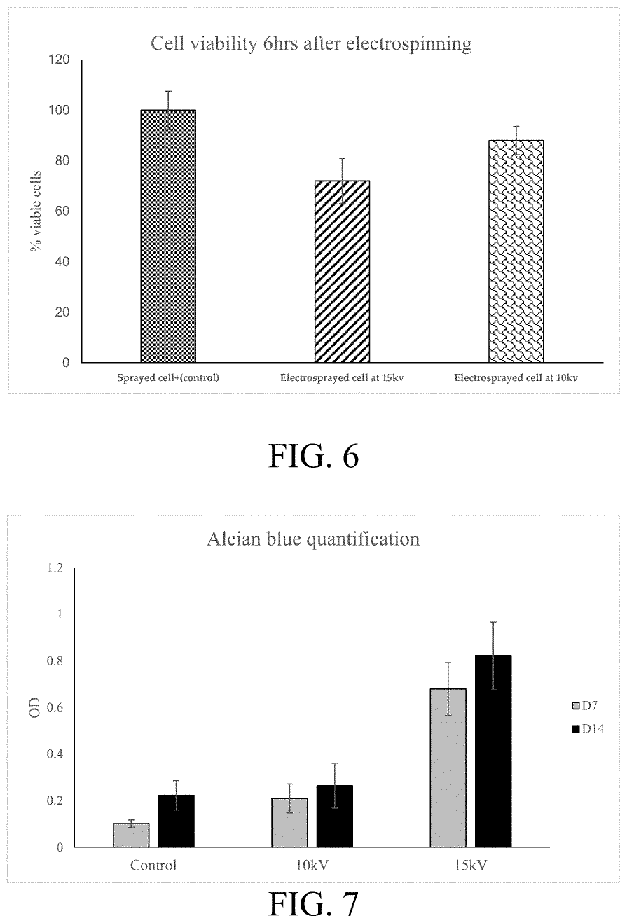

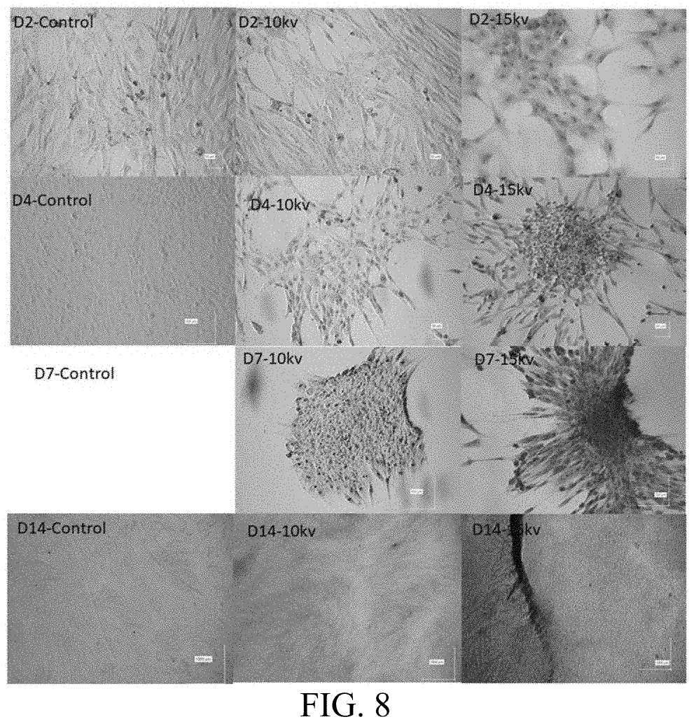

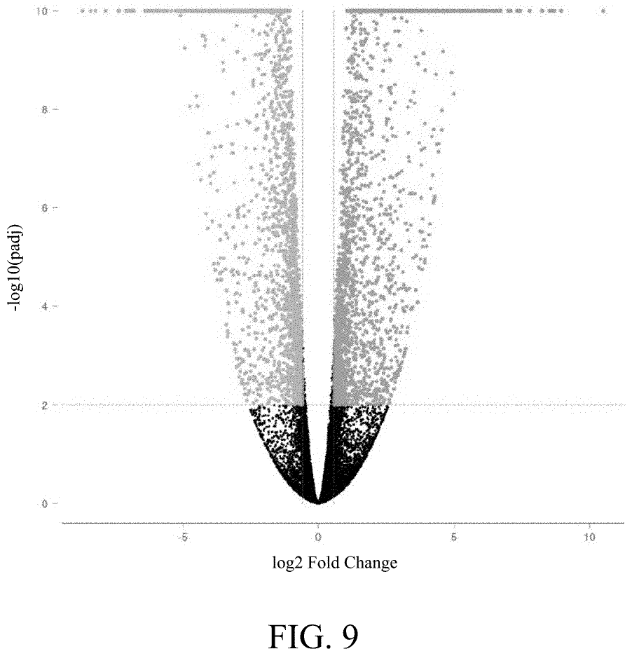

Using standard electrospinning conditions at 10 kV, cell viability using the pullulan/gelatin/cells formulation was preserving 90% viability, but electrospinning cells at 15 kV using the same polymers reduced the viability to about 70% (FIG. 6). Further, the effect of voltage on hADSC differentiation was examined to assess cell differentiation. Production of glycosaminoglycan (GAG) on the grown culture was analyzed based on alcian blue absorbance at 650 nm (FIGS. 7-8). Both the absorbance value of alcian blue stained cells in 10 kv and 15 kv increased gradually from day 2 to 14. The absorbance value in 15 kv was higher than any other group showing the maximum chondrogenesis and supporting the finding that electrospinning stem cells at increased voltage allows for the differentiation of the cells into, among other things, chondrocytes. Alcian blue staining was detected in the cells in the 15 kV group on D2-14. However this was not observed for the control or 10 kv group (FIG. 8). Further, RNA sequence analysis performed with electrospun cells 7-days after electrospinning revealed the presence of a number of TGF-β pathway genes that were upregulated (FIG. 9), including CDH2, TGFB2, FN1, CCL13, and IGF1.

With respect to the upregulated genes, TGFβs play critical roles in regulating chondrocyte differentiation from early to terminal stages, including condensation, proliferation, terminal differentiation, and maintenance of articular chondrocytes. There is a considerable amount of in vitro evidence to indicate that TGFβ signaling pathways promote mesenchymal condensation. In vitro data from D7 demonstrated that TGFβ1 induces mesenchymal cell condensation via up-regulation of N-cadherin and fibronectin (FN). GFβ1 treatment initiates chondrogenesis of mesenchymal progenitor cells. TGFβ2 and TGFβ3 are even more effective, causing a twofold greater accumulation of glycosaminoglycan. Insulin-like growth factor (IGF-1) is known as one of the important growth factors that can regulate the chondrogenic potential of cells and chondrocyte status and is upregulated in electrospun cells.

In summary, the data described in this Example 2 thus indicates that electric signaling is capable of providing a mesenchymal stem cell-based therapy for cartilage regeneration. Multiple previous studies have demonstrated the effects of various chemical factors, such as soluble growth factors, chemokines, and morphogens, on chondrogenesis. In particular, transforming growth factors (TGF-β) and bone morphogenetic proteins (BMPs) have been shown to play essential roles in the induction of chondrogenesis. Although growth factors have great therapeutic potential for cartilage regeneration, growth factor-based therapies have several clinical complications, including high dose requirements, low half-life, protein instability, higher costs, and adverse effects.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Barnes, C. P., Sell, S. A., Boland, E. D., Simpson, D. G. & Bowlin, G. L. Nanofiber technology: designing the next generation of tissue engineering scaffolds. Advanced drug delivery reviews 59, 1413-1433 (2007).
2. Glowacki, J. & Mizuno, S. Collagen scaffolds for tissue engineering. Biopolymers: Original Research on Biomolecules 89, 338-344 (2008).
3. Chan, B. & Leong, K. Scaffolding in tissue engineering: general approaches and tissue-specific considerations. European spine journal 17, 467-479 (2008).
4. Jun, I., Han, H.-S., Edwards, J. & Jeon, H. Electrospun fibrous scaffolds for tissue engineering: viewpoints on architecture and fabrication. International journal of molecular sciences 19, 745 (2018).
5. Frenot, A. & Chronakis, I. S. Polymer nanofibers assembled by electrospinning. Current opinion in colloid & interface science 8, 64-75 (2003).
6. Doshi, J. & Reneker, D. H. Electrospinning process and applications of electrospun fibers. Journal of electrostatics 35, 151-160 (1995).
7. Lu, T., Li, Y. & Chen, T. Techniques for fabrication and construction of three-dimensional scaffolds for tissue engineering. International journal of nanomedicine 8, 337 (2013).
8. Olson, J. L., Atala, A. & Yoo, J. J. Tissue engineering: current strategies and future directions. Chonnam medical journal 47, 1-13 (2011).
9. Gimble, J. M. & Guilak, F. Adipose-derived adult stem cells: isolation, characterization, and differentiation potential. Cytotherapy 5, 362-369 (2003).
10. Solchaga, L. A. et al. A rapid seeding technique for the assembly of large cell/scaffold composite constructs. Tissue engineering 12, 1851-1863 (2006).
11. Nosoudi, N., Holman, D; Karamched, S; Lei, Y; Rodriguez-Dévora, Jorge. Engineered Extracellular Matrix: Current Accomplishments and Future Trends. international journal of biomedical engineering and science 1 (2014).
12. Sauer, H., Rahimi, G., Hescheler, J. & Wartenberg, M. Effects of electrical fields on cardiomyocyte differentiation of embryonic stem cells. Journal of cellular biochemistry 75, 710-723 (1999).
13. Schwartz, L., da Veiga Moreira, J. & Jolicoeur, M. Physical forces modulate cell differentiation and proliferation processes. Journal of cellular and molecular medicine 22, 738-745 (2018).
14. Chen, W. Electroconformational denaturation of membrane proteins. Annals of the New York Academy of Sciences 1066, 92-105 (2006).
15. Li, D. & Xia, Y. Electrospinning of nanofibers: reinventing the wheel? Advanced materials 16, 1151-1170 (2004).
16. Townsend-Nicholson, A. & Jayasinghe, S. N. Cell electrospinning: a unique biotechnique for encapsulating living organisms for generating active biological microthreads/scaffolds. Biomacromolecules 7, 3364-3369 (2006).
17. Jayasinghe, S. N., Irvine, S. & McEwan, J. R. Cell electrospinning highly concentrated cellular suspensions containing primary living organisms into cell-bearing threads and scaffolds. (2007).
18. Bhattarai, D. P., Aguilar, L. E., Park, C. H. & Kim, C. S. A review on properties of natural and synthetic based electrospun fibrous materials for bone tissue engineering. Membranes 8, 62 (2018).
19. Babitha, S. et al. Electrospun protein nanofibers in healthcare: A review. International journal of pharmaceutics 523, 52-90 (2017).
20. Li, G., Fukunaga, S., Takenouchi, K. & Nakamura, F. Comparative study of the physiological properties of collagen, gelatin and collagen hydrolysate as cosmetic materials. International journal of cosmetic science 27, 101-106 (2005).
21. Huang, L., Nagapudi, K., P. Apkarian, R. & Chaikof, E. L. Engineered collagen-PEO nanofibers and fabrics. Journal of biomaterials science, Polymer edition 12, 979-993 (2001).
22. Nicholas, M. N., Jeschke, M. G. & Amini-Nik, S. Cellularized bilayer pullulan-gelatin hydrogel for skin regeneration. Tissue Engineering Part A 22, 754-764 (2016).
23. Frese, L., Dijkman, P. E. & Hoerstrup, S. P. Adipose tissue-derived stem cells in regenerative medicine. Transfusion Medicine and Hemotherapy 43, 268-274 (2016).
24. Mobini, S. et al. Fabrication and characterization of regenerated silk scaffolds reinforced with natural silk fibers for bone tissue engineering. Journal of Biomedical Materials Research Part A 101, 2392-2404 (2013).
25. Zhang, C., Gao, D., Ma, Y. & Zhao, X. Effect of gelatin addition on properties of pullulan films. Journal of food science 78, C805-C810 (2013).
26. Vacheethasanee, K., Wang, S., Qiu, Y. & Marchant, R. E. Poly (ethylene oxide) surfactant polymers. Journal of Biomaterials Science, Polymer Edition 15, 95-110 (2004).
27. Bulman, S. E. et al. Pullulan: a new cytoadhesive for cell-mediated cartilage repair. Stem cell research & therapy 6, 34 (2015).
28. Xu, Q., He, C., Xiao, C. & Chen, X. Reactive oxygen species (ROS) responsive polymers for biomedical applications. Macromolecular bioscience 16, 635-646 (2016).
29. Lim, L.-T., Mendes, A. C. & Chronakis, I. S. Electrospinning and electrospraying technologies for food applications. Advances in food and nutrition research 88, 167-234 (2019).
30. Topuz, F. & Uyar, T. Influence of Hydrogen-Bonding Additives on Electrospinning of Cyclodextrin Nanofibers. ACS Omega 3, 18311-18322 (2018).
31. Tsai, C. H., Lin, B. J. & Chao, P. H. G. α2β1 integrin and RhoA mediates electric field-induced ligament fibroblast migration directionality. Journal of Orthopaedic Research 31, 322-327 (2013).
32. Davidenko, N. et al. Evaluation of cell binding to collagen and gelatin: a study of the effect of 2D and 3D architecture and surface chemistry. Journal of Materials Science: Materials in Medicine 27, 148 (2016).
33. Meng, S. et al. Electrical stimulation in tissue regeneration. Applied biomedical engineering, 37-62 (2011).
34. Lanza, R., Langer, R. & Vacanti, J. P. Principles of tissue engineering. (Academic press, 2011).
35. Nasrollahzadeh, N., Applegate, L. A. & Pioletti, D. P. Development of an effective cell seeding technique: simulation, implementation, and analysis of contributing factors. Tissue Engineering Part C: Methods 23, 485-496 (2017).
36. Willerth, S. M. & Sakiyama-Elbert, S. E. Combining stem cells and biomaterial scaffolds for constructing tissues and cell delivery. StemJournal, 1-25 (2008).
37. Jun, I., Han, H.-S., Edwards, J. & Jeon, H. Electrospun fibrous scaffolds for tissue engineering: Viewpoints on architecture and fabrication. International journal of molecular sciences 19, 745 (2018).
38. Williams, G. R., Raimi-Abraham, B. T. & Luo, C. Nanofibres in Drug Delivery. (UCL Press, 2018).
39. Nosoudi, N. H., D; Karamched, S; Lei, Y; Rodriguez-Dévora, Jorge. Engineered Extracellular Matrix: Current Accomplishments and Future Trends. International Journal of Biomedical Engineering and Science (IJBES) 1 (2014).
40. Agarwal, S., Wendorff, J. H. & Greiner, A. Use of electrospinning technique for biomedical applications. Polymer 49, 5603-5621 (2008).
41. Nasim Nosoudi, Micah Jordan, J. M., Seba Aldabel, Chandra Hohne, Savannah Stultz, Alliah Turner, Paul Turner, Electrospinning live cells; a new step toward tissue engineering. accepted in Nature Scientific Reports.
42. Lode, A., Bernhardt, A. & Gelinsky, M. Cultivation of human bone marrow stromal cells on three-dimensional scaffolds of mineralized collagen: influence of seeding density on colonization, proliferation and osteogenic differentiation. Journal of tissue engineering and regenerative medicine 2, 400-407 (2008).
43. Bulman, S. E. et al. Pullulan: a new cytoadhesive for cell-mediated cartilage repair. Stem cell research & therapy 6, 34 (2015).
44. Xu, Q., He, C., Xiao, C. & Chen, X. Reactive oxygen species (ROS) responsive polymers
45. Zhang, C., Gao, D., Ma, Y. & Zhao, X. Effect of gelatin addition on properties of pullulan films. Journal of food science 78, C805-C810 (2013).
46. Shingel, K. I. Determination of structural peculiarities of dexran, pullulan and γ-irradiated pullulan by Fourier-transform IR spectroscopy. Carbohydrate Research 337, 1445-1451 (2002).
47. Khan, F. & Tanaka, M. Designing smart biomaterials for tissue engineering. International journal of molecular sciences 19, 17 (2018).
48. Menezes, J. & Luskin, M. B. Expression of neuron-specific tubulin defines a novel population in the proliferative layers of the developing telencephalon. Journal of Neuroscience 14, 5399-5416 (1994).
49. Bays, J. L. & DeMali, K. A. Vinculin in cell-cell and cell-matrix adhesions. Cellular and Molecular Life Sciences 74, 2999-3009 (2017).
50. Nosoudi, N., Yin, W. & Rubenstein, D. A. (Federation of American Societies for Experimental Biology, 2013).
51. Kamrannejad, M. M., Hasanzadeh, A., Nosoudi, N., Mai, L. & Babaluo, A. A. Photocatalytic degradation of polypropylene/TiO2 nano-composites. Materials Research 17, 1039-1046 (2014).
52. Nosoudi, N. et al. Calcium Phosphate/Etidronate Disodium Biocement: Etidronate, Retarder or Accelerator. Nano Biomedicine & Engineering 6 (2014).
53. Lei, Y., Sinha, A., Nosoudi, N., Grover, A. & Vyavahare, N. Hydroxyapatite and calcified elastin induce osteoblast-like differentiation in rat aortic smooth muscle cells. Experimental cell research 323, 198-208 (2014).
54. Park, J. et al. Small molecule-based lineage switch of human adipose-derived stem cells into neural stem cells and functional GABAergic neurons. Scientific reports 7, 10166 (2017).
55. Gao, S. et al. Differentiation of human adipose-derived stem cells into neuron-like cells which are compatible with photocurable three-dimensional scaffolds. Tissue Engineering Part A 20, 1271-1284 (2014).
56. Salehi, H., Amirpour, N., Niapour, A. & Razavi, S. An overview of neural differentiation potential of human adipose derived stem cells. Stem Cell Reviews and Reports 12, 26-41 (2016).
57. Dennis, G. et al. DAVID: database for annotation, visualization, and integrated discovery. Genome biology 4, R60 (2003).
58. Xiang, P. et al. The in vitro and in vivo biocompatibility evaluation of electrospun recombinant spider silk protein/PCL/gelatin for small caliber vascular tissue engineering scaffolds. Colloids and Surfaces B: Biointerfaces 163, 19-28 (2018).

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A cell scaffold, comprising an electrospun fiber and one or more live cells incorporated directly into the electropsun fiber.

2. The cell scaffold of claim 1, wherein the electrospun fiber comprises a protectant polymer.

3. The cell scaffold of claim 2, wherein the protectant polymer comprises a biocompatible water-soluble polymer.

4. The cell scaffold of claim 2, wherein the protectant polymer is selected from the group consisting of poly(ethylene glycol) (PEG), polyvinyl pyrrolidine (PVP), polyvinyl alcohol (PVA), polyacrylic acid (PAA), polyethylene oxide (PEO), N-(2-hydroxypropyl methacrylamide (HPMA), polyoxazoline, dextran, xanthan gum, hyaluronic acid (HA), albumin, starch, pullulan, gelatin, and combinations thereof.

5. The cell scaffold of claim 4, wherein the polymer comprises gelatin, pullulan, or a combination thereof.

6. The cell scaffold of claim 1, wherein the one or more cells are selected from the group consisting of a stem cell and a macrophage.

7. The cell scaffold of claim 6, wherein the stem cell is an adipose-derived stem cell, a mesenchymal stem cell, or a bone marrow-derived stem cell.

8. A method of making a cell scaffold including one or more live cells, comprising:
mixing one or more live cells with a protectant polymer and a biocompatible solvent to form a solution; and
electrospinning the solution at a working voltage of about 8 kV to about 35 kV.

9. The method of claim 8, wherein the protectant polymer comprises a biocompatible water-soluble polymer.

10. The method of claim 8, wherein the protectant polymer is selected from the group consisting of poly(ethylene glycol) (PEG), polyvinyl pyrrolidine (PVP), polyvinyl alcohol (PVA), polyacrylic acid (PAA), polyethylene oxide (PEO), N-(2-hydroxypropyl methacrylamide (HPMA), polyoxazoline, dextran, xanthan gum, hyaluronic acid (HA), albumin, starch, pullulan, gelatin, and combinations thereof.

11. The method of claim 10, wherein the polymer comprises gelatin, pullulan, or a combination thereof.

12. The method of claim 8, wherein the one or more cells are selected from the group consisting of a stem cell and a macrophage.

13. The method of claim 12, wherein the stem cell is an adipose-derived stem cell or a bone marrow-derived stem cell.

14. The method of claim 8, wherein the working voltage is about 8 kV to about 26 kV.

15. The method of claim 14, wherein the working voltage is about 8 kV or about 18 kV.

16. The method of claim 8, wherein the one or more cells comprise a stem cell, and wherein the working voltage is sufficient to differentiate the stem cell.

17. The method of claim 8, wherein the biocompatible solvent comprises phosphate-buffered saline or cell culture media.

18. The method of claim 8, further comprising mixing the solution with one or more additional polymers.

19. The method of claim 18, wherein the one or more additional polymers are selected from collagen, chitosan, poly(lactic-co-glycolic acid), and/or poly(ethylene oxide).

20. A method of making a cell scaffold including one or more live chondrocytes, comprising:
mixing one or more stem cells with a protectant polymer and a biocompatible solvent to form a solution; and
electrospinning the solution at a working voltage of about 10 kV to about 20 kV.

21. The method of claim 20, wherein the protectant polymer comprises a biocompatible water-soluble polymer.

22. The method of claim 20, wherein the protectant polymer is selected from the group consisting of poly(ethylene glycol) (PEG), polyvinyl pyrrolidine (PVP), polyvinyl alcohol (PVA), polyacrylic acid (PAA), polyethylene oxide (PEO), N-(2-hydroxypropyl methacrylamide (HPMA), polyoxazoline, dextran, xanthan gum, hyaluronic acid (HA), albumin, starch, pullulan, gelatin, and combinations thereof.

23. The method of claim 22, wherein the polymer comprises gelatin, pullulan, or a combination thereof.

24. The method of claim 20, wherein the stem cell is an adipose-derived stem cell or a bone marrow-derived stem cell.

25. The method of claim 20, wherein the biocompatible solvent comprises phosphate-buffered saline or cell culture media.
